(19) United States
(12) Patent Application Publication
(10) Pub. No.: US 2010/0047319 A1
(43) Pub. Date: Feb. 25, 2010

(54) Biodegradable Poly(Ester-Amide) And Poly(Amide) Coatings For Implantable Medical Devices With Enhanced Bioabsorption Times

(76) Inventors: Michael Huy Ngo, San Jose, CA (US); Syed F. A. Hossainy, San Jose, CA (US); Florencia Lim, Union City, CA (US); Mikael O. Trollsas, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 12/196,143

(22) Filed: Aug. 21, 2008

(51) Int. Cl. A61K 9/00

(52) U.S. Cl. 424/426; 424/400

(57) ABSTRACT

This invention is generally related to coating layers for implantable medical devices, such as drug delivery vascular stents, and methods of fabricating coated implantable medical devices. Specifically various embodiments of the present invention include methods of fabricating and modulating of coating layers to enhance bioabsorption. The coating layers include poly(ester-amide) and/or poly(amide) polymers.

### BACKGROUND

1. Field of the Invention

This invention is generally related to coatings for implantable medical devices, such as drug delivery vascular stents, and methods of fabricating coated implantable medical devices.

2. Description of the State of the Art

Percutaneous coronary intervention (PCI) is a procedure for treating heart disease. A catheter assembly having a balloon portion is introduced percutaneously into the cardiovascular system of a patient via the brachial or femoral artery. The catheter assembly is advanced through the coronary vasculature until the balloon portion is positioned across the occlusive lesion. Once in position across the lesion, the balloon is inflated to a predetermined size to radially compress the atherosclerotic plaque of the lesion to remodel the lumen wall. The balloon is then deflated to a smaller profile to allow the catheter to be withdrawn from the patient's vasculature.

Problems associated with the above procedure include formation of intimal flaps or torn arterial linings which can collapse and occlude the blood conduit after the balloon is deflated. Moreover, thrombosis and restenosis of the artery may develop over several months after the procedure, which may require another angioplasty procedure or a surgical by-pass operation. To reduce the partial or total occlusion of the artery by the collapse of the arterial lining and to reduce the chance of thrombosis or restenosis, a stent is implanted in the artery to keep the artery open.

Drug delivery stents have reduced the incidence of in-stent restenosis (ISR) after PCI (see, e.g., Serruys, P. W., et al., J. Am. Coll. Cardiol. 39:393-399 (2002)), which has plagued interventional cardiology for more than a decade. However, ISR still poses a significant problem given the large volume of coronary interventions and their expanding use. The pathophysiological mechanism of ISR involves interactions between the cellular and acellular elements of the vessel wall and the blood. Damage to the endothelium during PCI constitutes a major factor for the development of ISR (see, e.g., Kipshidze, N., et al., J. Am. Coll. Cardiol. 44:733-739 (2004)).

The embodiments of the present invention relate to drug delivery stents, methods of fabricating drug delivery stents, as well as others embodiments that are apparent to one having ordinary skill in the art.

### SUMMARY

Various embodiments of the present invention include methods of modulating the in vivo absorption rate of a coating layer on an implantable medical device comprising a poly(ester-amide) (PEA) polymer, poly(amide) (PA) polymer, or a combination thereof, and a drug where the coating layer comprises a polymer phase and a dispersed drug phase. The methods include the operations of: a) causing faster and greater water ingress into the coating layer; b) increasing fraction of interfacial area of the polymer with the dispersed drug phase; and/or c) increasing the surface area of the coating layer or the interfacial area of the polymer phase with the dispersed drug phase. The coating layer completely degrades or substantially degrades in 12 months or fewer after implantation of the implantable medical device.

In some embodiments, the implantable medical device is a stent.

In some embodiments, causing faster and greater water ingress into the coating layer includes adding a non-therapeutic soluble component to the coating layer.

In some embodiments, the coating layer completely or substantially degrades in 6 months or fewer after implantation.

In some embodiments, the coating layer has degraded by about 50% at 3 months after implantation, or in less than 3 months after implantation.

In some embodiments, increasing the fraction of interfacial area of the polymer with the dispersed drug phase comprises using a drug to polymer ratio in the range of about 1:1 to about 1:7, and/or using a soluble component to polymer ratio in the range of about 1:1 to about 1:7.

In some embodiments, increasing fraction of interfacial area of the polymer with the dispersed drug phase comprises. In some embodiments, increasing the surface area of the coating layer or the interfacial area of the polymer phase with the dispersed drug phase comprises applying the coating layer such that the domain size of the domains of the dispersed drug phase are between about 100 nm and about 1-2 μm.

In some embodiments, the coating layer is a solid solution.

In some embodiments, the method modulates a coating layer on an implantable medical device, and the coating layer comprises a poly(ester-amide) (PEA) polymer, poly(amide) (PA) polymer, or a combination thereof, and a drug, wherein the PEA and/or PA polymer is a polymer of the formula:

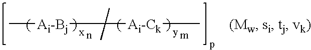

wherein:

i is an integer from 1 to 10, inclusive;

j is an integer from 0 to 10, inclusive;

k is an integer from 0 to 15, inclusive;

xₙ is an integer from 0 to 100, inclusive;

yₘ is an integer from 0 to 150, inclusive;

p is an integer from 2 to about 4500;

M_{w} is from about 10,000 to about 1,000,000 Da;

sᵢ is a number from 0 to 0.5, inclusive;

tⱼ is a number from 0 to 0.5, inclusive;

vₖ is a number from 0 to 0.5, inclusive;

with the proviso that

Σᵢ sᵢ+Σⱼ tⱼ+Σₖ vₖ=1.0;

Σᵢ sᵢ=Σⱼ tⱼ+Σₖ vₖ=0.5;

Σᵢ sᵢ>0;

Σⱼ tⱼ>0 or Σₖ vₖ>0;

each Aᵢ has the chemical structure:

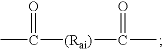

each Bⱼ has the chemical structure

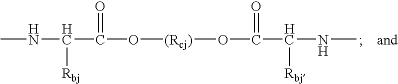

each Cₖ has the chemical structure:

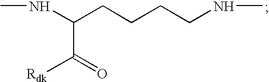

wherein:

each R_{bj}, and R_{bj′} are independently selected from the group consisting of hydrogen and (C1-C4)alkyl, wherein the alkyl group is optionally substituted with a moiety selected from the group consisting of —OH, —SH, —SeH, —C(O)OH, —NHC(NH)NH₂,

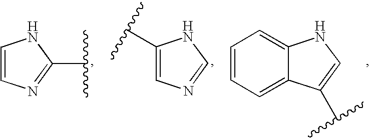

phenyl and

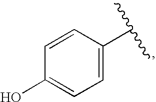

or one or more of R_{bj} and R_{bj′} may form a bridge between the carbon to which it is attached and the adjacent nitrogen, the bridge comprising —CH₂CH₂CH₂—. Each Rₐᵢ and each R_{cj} are independently selected from the group consisting of (C1-C12)alkyl, (C2-C12)alkenyl, (C3-C8)cycloalkyl, —(CH₂CH₂O)_{q}CH₂CH₂— wherein q is an integer from 1 to 10, inclusive, and

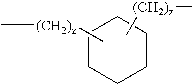

where z is 0, 1, or 2. Each R_{dk} is selected from the group consisting of —H, —OH, —O(C1-C20)alkyl, —O(C1-C20)alkenyl and —O(CH₂CH₂O))_{w}CH₂CH₂ORₑₖ, wherein w is an integer from 1 to 600, inclusive; and Rₑₖ is selected from the group consisting of hydrogen, —C(O)CH═CH₂ and —C(O)C(CH₃)═CH₂; and, each Rₐᵢ corresponds to the i^{th} Aᵢ group, each R_{bj}, R_{bj′}, and R_{cj} corresponds to the j^{th} Bⱼ group, and each R_{dk} and optionally Rₑₖ correspond to the k^{th} Cₖ group.

In some embodiments Rₐᵢ, R_{cj}, and R_{dk} may be a hydrocarbon of 2 to 20 carbon atoms containing one or more double bonds and one or more triple bonds.

In some embodiments, the polymer is of the formula above wherein i=1, j=2, k=0, and each of Rₐ₁ is selected from the group consisting of —(CH₂)₆—, —(CH₂)₇—, —(CH₂)₈—, —(CH₂)₉—, and —(CH₂)₁₀—; each of R_{b1}, R_{b1′}, R_{b2} and R_{b2′} are the same, and are selected from the group consisting of —CH₂—CH(CH₃)₂ and —CH₃; R_{c1} is selected from the group consisting of —(CH₂)₄—, —(CH₂)₅—, —(CH₂)₆—, —(CH₂)₇—, and —(CH₂)₈—; and R_{c2} is selected from the group consisting of

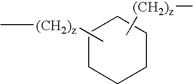

where z is 0, 1, or 2.

In some embodiments, the coating layer includes a PEA polymer which is a random co-polymer of the following formula [-(A₁-B₁)-/-(A₁-B₂)-]ₚ(M_{w}, t₁, t₂, s₁):

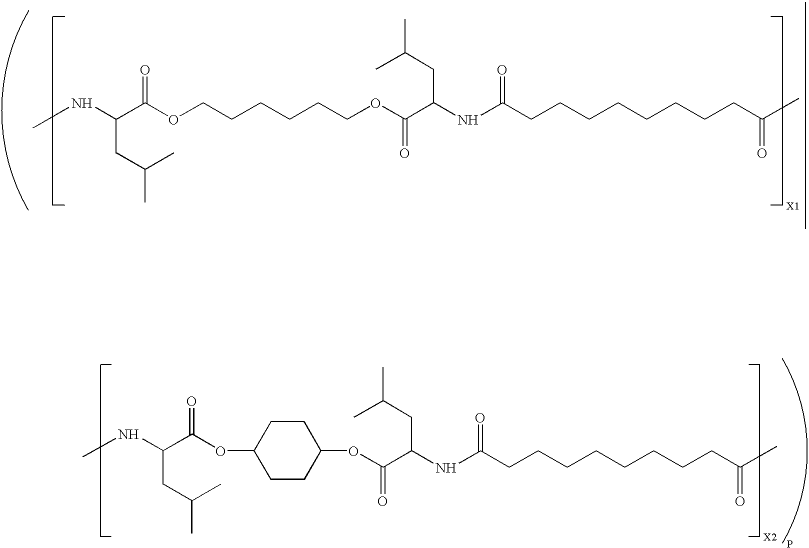

in which s₁ is 0.5, and each of t₁ is between 0.125 and 0.375 and t₂ is 0.5−t₁, thus the mole fractions of the two constitutional units, represented here by subscripts X1 and X2, range from about 0.25 to about 0.75, and p is the total number of X1 and X2 units on average, per polymer chain and ranges from 2 to about 4500. In a preferred embodiment, t₁ and t₂ are about each 0.25, and thus the mole fraction of the X1 and X2 units is about 0.5. The two groups do not necessarily alternate regularly as this is a random copolymer, and there are multiple X1 and X2 groups per polymer chain. There may be large variations in the length of the polymer chains.

In some embodiments, the coating layer includes a non-therapeutic soluble component in addition to or instead of a drug.

Various embodiments of the present invention include methods of fabricating an implantable medical device coated with a bioabsorbable coating layer. The methods include the operations of: applying to an implantable medical device a coating layer comprising a PEA polymer, a PA polymer, or a combination thereof, and a soluble component with a soluble component to polymer ratio between 1:1 to 1:7, and a coating layer thickness between about 2 μm and about 10 μm. The polymer in the coating layer, or the coating layer, is substantially absorbed, or completely absorbed, in vivo in 12 months or fewer after implantation. The soluble component may be a drug in some embodiments or the soluble component may be a non-therapeutic soluble component in some embodiments.

Various embodiments of the present invention include a coating layer on a substrate. The coating layer includes a drug and a PEA polymer, a PA polymer, or a combination thereof, with the ratio of drug to polymer from about 1:3 to about 1:5. The coating layer thickness is between 2 μm to about 10 μm. The polymer mass in the coating layer 3 months after implantation of the substrate is about 50% or less of the initial polymer mass in the coating layer where the reduction is due to in vivo absorption. In some embodiments, the PEA polymer, PA, polymer, or combination thereof, may be any of the PEA and/or PA polymers described in the prior paragraphs.

Various embodiments of the present invention also encompass a coated implantable medical device, such as a stent, that is coated with the coating layer described in the previous paragraph.

Various embodiments of the present invention include methods of fabricating an implantable medical device coated with a bioabsorbable coating layer. The methods include the operations of: applying to an implantable medical device a coating layer comprising a the PEA polymer, PA, polymer, or combination thereof, and a soluble component wherein the soluble component to polymer ratio is between 1:1 to 1:7; and wherein the coating layer thickness is between about 2 μm and 10 μm; and determining the in vivo bioabsorption time of the coating layer. If the in vivo bioabsorption time is too long, increasing the ratio of soluble component to polymer, decreasing the coating layer thickness, and/or decreasing the domain size of the soluble component. If the in vivo bioabsorption time is too short, decreasing the ratio of soluble component to polymer, increasing the coating layer thickness, and/or increasing the domain size of the soluble component.

### BRIEF DESCRIPTION OF THE DRAWINGS

FIG. 1 is a poly(ester-amide) polymer utilized in an exemplary embodiment of the present invention.

FIGS. 2 and 3 depict stents coated with exemplary coating layers of the present invention.

FIG. 4 is an illustration of the cumulative release profiles of everolimus from stents coated with exemplary coating layers of the present invention.

FIG. 5 is a graph modeling the in vivo bioabsorption of some exemplary coating layers of
the present invention compared to the modeled in vivo bioabsorption of a poly(D,L lactide) coating layer.

FIG. 6 is a graph of the mass of polymer degraded at 3 months versus the mass fraction of polymer in the coating layer.

### DETAILED DESCRIPTION

Provided herein are various embodiments of methods of fabricating a bioabsorbable coating layer with an in vivo bioabsorption of about 12 months or fewer. In addition, provided herein are various embodiments of methods for enhancing the in vivo bioabsorption of such a coating layer. The various coating layers include a PEA polymer, a PA polymer, or combinations thereof, and optionally, a drug. Such coating layers may also include soluble components.

It has surprisingly been found that the in vivo bioabsorption is a function of the mass ratio of the drug to the polymer in a coating layer comprising a polymer which is a PEA polymer and/or a PA polymer. These bioabsorption rates are not predicted by conventional in vitro degradation times. Thus, the in vivo absorption of the coating layer, or the polymer included in the coating layer, may be enhanced by increasing the mass ratio of the drug to the polymer. The thickness plays a role in the degradation rate as the removal of products of degradation is quicker for thinner coating layers compared to a thicker coating layer. However, for the first estimate, the effect of coating layer thickness does not need to be considered.

Without being bound by theory, it is believed that the in vivo bioabsorption is a function of the rate of fluid or water ingress into the coating layer, and the surface area or interfacial area of the coating layer exposed to fluids. The interfacial surface area that is the area of the coating layer directly in contact with bodily fluids when implanted, is increased as the drug, and/or other soluble components, are released and/or diffuse out of the coating layer. Thus, the interfacial area of the coating layer is expected to increase as the drug to polymer ratio increases and/or ratio of soluble components (including drug, if classified as a soluble component) to the polymer increases. It is also believed that the increased interfacial surface area increases, the mass transport coefficient for the products of the degradation. A higher mass transfer coefficient for the degradation products results in more rapid removal of these products from the coating layer. Thus, a higher surface area would lead to a higher in vivo absorption rate.

It was also found that the in vitro degradation of the coating layer comprising a poly(ester-amide) and a drug did not correspond with the in vivo bioabsorption rate. The in vivo bioabsorption rate was found to be significantly higher than the in vitro degradation rate. It is believed that the higher in vivo bioabsorption compared to the in vitro degradation is due to a cell-mediated process occurring in vivo. The in vitro degradation is due to hydrolysis, a chemical reaction. However, the rate of hydrolysis is lower than the in vivo biodegradation rate indicating that hydrolysis is not the only process responsible for polymer degradation in vivo. It is believed that such results also apply to the PA polymers described herein.

Therefore, by adjusting the ratio of drug to polymer in the coating layer, the in vivo degradation rate may be modulated. More generally, the ratio of soluble component to polymer in a coating layer comprising a PEA polymer, PA polymer, or a combination thereof controls the rate of in vivo bioabsorption. In addition, the size of the domains of the soluble components is believed to have an effect on the degradation rate. Thickness also impacts the rate of removal of the products of biodegradation with the removal rate being higher for thinner coating layers as the diffusion distance is shorter. Thus, the ratio of drug and/or other soluble components to polymer, coating layer thickness, as well as the domain size of the drug may all be adjusted to modulate the in-vivo absorption.

Although the discussion that follows may refer to a stent as an exemplary embodiment of a medical device that may be used with the various embodiments of the present invention, the various embodiments of the present invention are not limited to use with stents. Also, although the various embodiments may refer to a coating layer with “a polymer,” “a drug,” or “a soluble component,” it is understood that the various embodiments of the present invention encompass one or more polymers, one or more drugs, and/or one or more soluble components.

### Definitions

As used herein, unless specified otherwise, any words of approximation such as without limitation, “about,” “essentially,” “substantially” and the like mean that the element so modified need not be exactly what is described but can vary from the description by as much as ±15% without exceeding the scope of this invention.

As used herein, “therapeutic agent,” “drug,” “active agent,” and “bioactive agent,” which will be used interchangeably, refer to any substance that, when administered in a therapeutically effective amount to a patient suffering from a disease or condition, has a therapeutic beneficial effect on the health and well-being of the patient. A therapeutic beneficial effect on the health and well-being of an individual includes, but it not limited to: (1) curing the disease or condition; (2) slowing the progress of the disease or condition; (3) causing the disease or condition to retrogress; or, (4) alleviating one or more symptoms of the disease or condition.

As used herein, a drug also includes any substance that when administered to an individual, known or suspected of being particularly susceptible to a disease, in a prophylactically effective amount, has a prophylactic beneficial effect on the health and well-being of the individual. A prophylactic beneficial effect on the health and well-being of an individual includes, but is not limited to: (1) preventing or delaying on-set of the disease or condition in the first place; (2) maintaining a disease or condition at a retrogressed level once such level has been achieved by a therapeutically effective amount of a substance, which may be the same as or different from the substance used in a prophylactically effective amount; or, (3) preventing or delaying recurrence of the disease or condition after a course of treatment with a therapeutically effective amount of a substance, which may be the same as or different from the substance used in a prophylactically effective amount, has concluded.

As used herein, “therapeutic agent,” “drug,” “active agent,” and “bioactive agent” also encompass pharmaceutically acceptable salts, esters, amides, prodrugs, active metabolites, analogs, and the like.

As used herein, a “polymer” is a molecule made up of the repetition of a simpler unit, herein referred to as a constitutional unit. The constitutional units themselves can be the product of the reactions of other compounds. A polymer may comprise one or more types of constitutional units. As used herein, the term polymer refers to a molecule comprising 2 or more constitutional units. A “monomer” is compound which may be reacted to form a polymer, or part of a polymer, but is not itself the repetition of a simpler unit. A monomer is not equivalent to a constitutional unit, but is related to a constitutional unit. As a non-limiting example, CH₂═CH₂ or ethylene is reacted to form polyethylene, such as CH₃(CH₂)₅₀₀CH₃, for which the constitutional unit is —CH₂—CH₂— and for which ethylene CH₂═CH₂ would be considered to be a monomer. Thus, the monomer may contain bonds, and/or atoms that are lost once the polymer is formed, and therefore the monomer and constitutional unit are not identical, but are related. Polymers may be straight or branched chain, star-like or dendritic, or a polymer may be attached (grafted) onto another polymer. Polymers may be cross-linked to form a network.

As used herein, the term “oligomer” refers to a molecule including fewer than 20 constitutional units, and as used herein, is a subset of polymers.

As used herein, “copolymer” refers to a polymer which includes more than one type of constitutional unit (or made by reaction of more than one type of monomer).

As used herein, the term “soluble components” refers to compounds which dissolve or diffuse, or are released, or substantially released, from a coating layer comprising a polymer on a time frame that is much shorter than the time frame for the polymer to biodegrade, or to substantially biodegrade. A time frame that is much shorter is about 35% or less of the time frame for the polymer to biodegrade. Such soluble components may include, without limitation, drugs, oligomers, some polymers, and lower molecular weight compounds such as sugars. Not all drugs, polymers, and/or oligomers are necessarily “soluble components,” but some compounds in each of the three groups may be categorized as “soluble components.” For polymers, the categorization may be dependent upon molecular weight. It is expected that many drugs may be categorized as “soluble components” with respect to the coating layers of the various embodiments of the present invention. Although the term “soluble” is used, there is no specific aqueous solubility requirement for these compounds to be categorized as “soluble components.”

As used herein, “non therapeutic soluble compounds,” are those soluble compounds which are not classified as “drugs.”

As used herein, a “poly(ester-amide)” refers to a polymer that has in its backbone structure both ester and amide bonds.

As used herein, a “poly(amide)” refers to a polymer that has in its backbone structure amide bonds.

As used herein, the terms “biodegradable,” “bioerodable,” “bioabsorbable,” and “degraded,” are used interchangeably, and refer to polymers, coating layers, and materials, that are capable of being completely or substantially completely, degraded, dissolved, and/or eroded over time when exposed to physiological conditions (pH, temperature, and fluid or other environment), and can be gradually resorbed, absorbed and/or eliminated by the body, or that can be degraded into fragments that can pass through the kidney membrane of an animal (e.g., a human). Conversely, a “biostable” polymer, coating layer, or material, refers to a polymer, coating layer or material that is not biodegradable.

As used herein, “degradation time,” “biodegradation time,” and “absorption time,” are used interchangeably, and refer to the time for a biodegradable material implanted in a host animal to completely bioabsorb in vivo or substantially bioabsorb in vivo, unless the context clearly indicates otherwise, or it is expressly stated otherwise. For example, in vitro degradation expressly refers to an in vitro as opposed to in vivo measurement. In some cases, some residue may remain.

As used herein, “substantially degrade,” “substantially bioabsorb,” and “substantially biodegrade,” refers to degradation, or loss of mass, of about 80% or more.

As used herein, the measurement or determination of “bioabsorption” or “biodegradation” of a coating or a coating layer will refer to the mass loss of the non-soluble components in the coating or coating layer. Thus, if the coating layer includes soluble components, such as, without limitation, a drug, the mass loss of these components is not included in the mass loss of the coating layer for calculation of the % biodegradation or % bioabsorption. In other words, the % biodegradation is calculated with reference to only the non-soluble components, generally the one or more polymers in the coating layer.

As used herein, the “percolation threshold” is the point at which domains of one phase in a multiple phase system begin to connect and form an interconnected network of the phase within the multiple phase system. The percolation threshold is the point at which the one phase can form its own channel for diffusion through interconnected domains. Percolation thresholds are generally expressed as a volume fraction and are a function of the domain size and shape for each of the phases in the multiple phase system.

As used herein, “substantially released” refers to a cumulative release of the drug of about 80% or more.

### Drugs in the Coating Layer

Some embodiments of the present invention may include one or more drugs in the coating layer. Some of the drugs may also be categorized as “soluble components.” Some of the drugs may not be categorized as “soluble components.”

Some embodiments include an antiproliferative drug. The term “anti-proliferative” as used herein, refers to an agent that works to block the proliferative phase of acute cellular rejection. Examples of anti-proliferative agents include rapamycin and its functional or structural derivatives including without limitation, Biolimus A9 (Biosensors International, Singapore), deforolimus, AP23572 (Ariad Pharmaceuticals), tacrolimus, temsirolimus, pimecrolimus, zotarolimus (ABT-578), 40-O-(2-hydroxy)ethyl-rapamycin (everolimus), 40-O-(3-hydroxypropyl)rapamycin, 40-O-[2-(2-hydroxy)ethoxy]ethyl-rapamycin, 40-O-tetrazole-rapamycin, 40-O-tetrazolylrapamycin, 40-epi-(N1-tetrazolyl)-rapamycin, and the functional or structural derivatives of everolimus. Other examples include paclitaxel and its functional and structural derivatives. An example of a paclitaxel derivative is docetaxel.

Everolimus and zotarolimus are drugs which may also be categorized as a “soluble component.”

Active agents other than anti-proliferative drugs may be used. Other examples of suitable active agents, include, but are not limited to, synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules that bind to complementary DNA to inhibit transcription, and ribozymes. Some other examples of other active agents include antibodies, receptor ligands such as the nuclear receptor ligands estradiol and the retinoids, enzymes, adhesion peptides, blood clotting factors, inhibitors or clot dissolving drugs such as streptokinase and tissue plasminogen activator, antigens for immunization, hormones and growth factors, oligonucleotides such as antisense oligonucleotides, ribozymes and retroviral vectors for use in gene therapy, and genetically engineered endothelial cells. Other active agents include heparin, fragments and derivatives of heparin, glycosamino glycan (GAG), GAG derivatives, alpha-interferon, and thiazolidinediones (glitazones). The drugs could be designed, e.g., to inhibit the activity of vascular smooth muscle cells. They could be directed at inhibiting abnormal or inappropriate migration and/or proliferation of smooth muscle cells to inhibit restenosis.

Examples of drugs that may be suitable for use in the various embodiments of the present invention, depending, of course, on the specific disease being treated, include, without limitation, anti-restenosis, pro-proliferative, anti-inflammatory, anti-neoplastic, antimitotic, anti-platelet, anticoagulant, antifibrin, antithrombin, cytostatic, antibiotic, anti-enzymatic, anti-metabolic, angiogenic, cytoprotective, angiotensin converting enzyme (ACE) inhibiting, angiotensin II receptor antagonizing and/or cardioprotective drugs.

In some embodiments, an antiproliferative drug may be a natural proteineous substance such as a cytotoxin or a synthetic molecule. Examples of antiproliferative substances, which may be classified as anti-proliferative active agents, include, without limitation, actinomycin D or derivatives and analogs thereof (manufactured by Sigma-Aldrich, or COSMEGEN available from Merck) (synonyms of actinomycin D include dactinomycin, actinomycin IV, actinomycin I₁, actinomycin X₁, and actinomycin C₁); all taxoids such as taxols, docetaxel, and paclitaxel and derivatives thereof; the macrolide antibiotic rapamycin (sirolimus) and its derivatives (as outlined above); all olimus drugs; FKBP-12 mediated mTOR inhibitors, prodrugs thereof, co-drugs thereof, and combinations thereof Additional examples of cytostatic or antiproliferative drugs include, without limitation, angiopeptin, and fibroblast growth factor (FGF) antagonists.

Examples of anti-inflammatory drugs include both steroidal and non-steroidal (NSAID) anti-inflammatories such as, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone, dexamethasone dipropionate, dexamethasone acetate, dexmethasone phosphate, momentasone, cortisone, cortisone acetate, hydrocortisone, prednisone, prednisone acetate, betamethasone, betamethasone acetate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus and pimecrolimus.

Alternatively, the anti-inflammatory drug can be a biological inhibitor of pro-inflammatory signaling molecules. Anti-inflammatory biological active agents include antibodies to such biological inflammatory signaling molecules.

Examples of antineoplastics and antimitotics include, without limitation, paclitaxel, docetaxel, methotrexate, azathioprine, vincristine, vinblastine, fluorouracil, doxorubicin hydrochloride and mitomycin.

Examples of anti-platelet, anticoagulant, antifibrin, and antithrombin drugs include, without limitation, heparin, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as ANGIOMAX® (bivalirudin), calcium channel blockers such as nifedipine, colchicine, fish oil (omega 3-fatty acid), histamine antagonists, lovastatin, monoclonal antibodies such as those specific for Platelet-Derived Growth Factor (PDGF) receptors, nitroprusside, phosphodiesterase inhibitors, prostaglandin inhibitors, suramin, serotonin blockers, steroids, thioprotease inhibitors, triazolopyrimidine, nitric oxide or nitric oxide donors, super oxide dismutases, super oxide dismutase mimetic and 4-amino-2,2,6,6-tetramethylpiperidine-1-oxyl (4-amino-TEMPO).

Examples of ACE inhibitors include, without limitation, quinapril, perindopril, ramipril, captopril, benazepril, trandolapril, fosinopril, lisinopril, moexipril and enalapril.

Examples of angiogensin II receptor antagonists include, without limitation, irbesartan and losartan.

Other drugs include anti-infectives such as antiviral drugs; analgesics and analgesic combinations; anorexics; antihelmintics; antiarthritics, antiasthmatic drugs; anticonvulsants; antidepressants; antidiuretic drugs; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary vasodilators; peripheral and cerebral vasodilators; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and restenoic reducing drugs.

Some active agents may fall into more than one of the above mentioned categories.

### Soluble Components in the Coating Layer

Some embodiments of the present invention encompass the inclusion of one or more soluble components in the coating layer. In some embodiments, the drug utilized may also be categorized as “soluble component,” while in other embodiments the drug utilized may not be categorized as a “soluble component.” Those soluble components which are not classified as drugs may be referred to as “non-therapeutic soluble components.”

Some non-limiting examples of soluble components include representative hydrophilic polymers, typically of a lower molecular weight than the PEA and/or PA polymer, such as, without limitation, polymers and co-polymers of PEG acrylate (PEGA), PEG methacrylate, 2-methacryloyloxyethylphosphorylcholine (MPC) and n-vinyl pyrrolidone (VP), polymers and co-polymers of carboxylic acid bearing monomers such as methacrylic acid (MA), acrylic acid (AA), hydroxyl bearing monomers such as HEMA, hydroxypropyl methacrylate (HPMA), hydroxypropylmethacrylamide, and 3-trimethylsilylpropyl methacrylate (TMSPMA), poly(ethylene glycol) (PEG), poly(propylene glycol), SIS-PEG, polystyrene-PEG, polyisobutylene-PEG, PCL-PEG, PLA-PEG, PMMA-PEG, PDMS-PEG, PVDF-PEG, PLURONIC™ surfactants (polypropylene oxide-co-polyethylene glycol), poly(tetramethylene glycol), a block copolymer having flexible poly(ethylene glycol) and poly(butylene terephthalate) blocks (PEGT/PBT) (e.g., PolyActive™),

poly(L-lysine-ethylene glycol) (PLL-g-PEG), poly(L-g-lysine-hyaluronic acid) (PLL-g-HA), poly(L-lysine-g-phosphoryl choline) (PLL-g-PC), poly(L-lysine-g-vinylpyrrolidone) (PLL-g-PVP), poly(ethylimine-g-ethylene glycol) (PEI-g-PEG), poly(ethylimine-g-hyaluronic acid) (PEI-g-HA), poly(ethylimine-g-phosphoryl choline) (PEI-g-PC), and poly(ethylimine-g-vinylpyrrolidone) (PEI-g-PVP), PLL-co-HA, PLL-co-PC, PLL-co-PVP, PEI-co-PEG, PEI-co-HA, PEI-co-PC, and PEI-co-PVP, poly(vinyl pyrrolidone) and hydroxyl functional poly(vinyl pyrrolidone), polyalkylene oxides, dextran, dextrin, sodium hyaluronate, hyaluronic acid, elastin, chitosan, acrylic sulfate, acrylic sulfonate, acrylic sulfamate, methacrylic sulfate, methacrylic sulfonate, methacrylic sulfamate and combinations thereof. PolyActive™ is intended to include AB, ABA, BAB copolymers having such segments of PEG and PBT (e.g., poly(ethylene glycol)-block-poly(butyleneterephthalate)-block poly(ethylene glycol) (PEG-PBT-PEG).

Other soluble components include low molecular weight compounds such as sugars, or starches.

In some embodiments, the soluble components utilized may have a high osmotic effect. Non-limiting examples include sodium chloride and sodium carbonate.

### Polymers in the Coating Layer

Various embodiments of the present invention utilize PEA and/or PA polymers. However, additional polymers of other types may also be included along with a PEA and/or a PA polymer. The various embodiments of the present invention encompass blends of polymers as well as the use of one type of polymer.

Various embodiments of the present invention include poly(ester-amide) and poly(amide) polymers having the following generic formula:

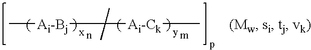

wherein the constitutional units are represented by Aᵢ-Bⱼ and Aᵢ-Cₖ where the Aᵢ and Bⱼ react to form the constitutional unit represented by Aᵢ-Bⱼ and Aᵢ and Cₖ react to form the constitutional unit represented by Aᵢ-Cₖ. The Aᵢ groups are derived from diacids, and the Bⱼ groups are derived from diamino esters. The group Cₖ is a lysine group. Thus, each Aᵢ has the chemical structure:

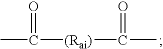

each Bⱼ has the chemical structure

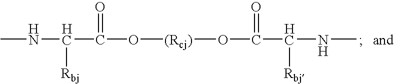

each Cₖ has the chemical structure:

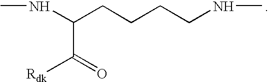

As noted above, the constitutional units themselves may be the products of the reactions of other compounds. For example, without limitation, a Bⱼ group above can comprise the reaction of an amino acid,

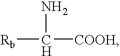

with a diol, HO—(R_{c})—OH, to give a diamino ester,

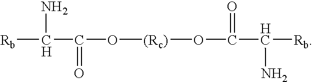

The diamino ester may be further reacted with a diacid,

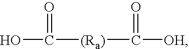

to give the constitutional unit, represented by Aᵢ-Bⱼ. The amine group, the carboxylic acid group or the hydroxyl group may be “activated,” i.e., rendered more chemically reactive, to facilitate the reactions if desired; such activating techniques are well-known in the art and the use of any such techniques is within the scope of this invention.

While any amino acid may be used to construct a polymer of this invention, particularly useful amino acids are the so-called essential amino acids of which there currently 20: alanine, arginine, asparagine, aspartic acid, cysteine, glutamic acid, glutamine, glycine, histidine, isoleucine, leucine, lysine, methionine, phenyl alanine, proline, serine, threonine, tryptophan, tyrosine and valine. More recently selenoadenine has been found to be incorporated into a number of proteins and is included as a particularly useful amino acid of this invention. In naturally-occurring biological proteins, these amino acids appear as the l-enantiomeric isomers but for the purposes of this invention they may be used as their l- or d-enantiomers or as racemic mixtures.

On the lysine unit, represented by Cₖ, the R_{dk} can be a drug, a peptide (which may a drug or a targeting moiety), a polymer, an oligomer, or another type of functional group. The polymer or oligomer may be hydrophilic or hydrophobic. R_{dk} may also be a protective group to prevent the pendent acid functionality from participating in the polymerization reaction.

The linkage used to directly attach R_{dk} to the carbonyl of the lysine may be an ester, a thioester, an amide, an anhydride, or an imide or R_{dk} may be connected to the carbonyl through a spacer such as, without limitation, a C1-C12 alkyl or a poly(alkylene oxide) such as poly(ethylene glycol) or poly(propylene oxide).

As noted above, each Aᵢ and Bⱼ represents one or more different groups derived from diacids or derived from diamino esters, respectively, which may react to form the constitutional units, where i represents the i^{th} type of Aᵢ group, j represents the j^{th} type of Bⱼ group, and k represents the k^{th} type of Cₖ groups. Each polymer may have from 1 to 10 Aᵢ groups. Similarly, each polymer may have from 0 to 10 Bⱼ groups, and from 0 to 15 Cₖ groups. A particular polymer may have fewer than the maximum, or 10, different Aᵢ groups. Thus if i=3, there is an A₁, A₂ and A₃ group. Similarly a particular polymer may have fewer different Bⱼ groups than the maximum, 10, and a particular polymer may have less than the maximum number of types of Cₖ groups possible, that is 15. Therefore, if j=2, there is a B₁ and a B₂ group, and if k=0 there are no Cₖ groups. There must be at least one Aᵢ group, or i is at least one (1). In addition, there must be at least one Bⱼ group, or at least one Cₖ group, or in other words, both j and k cannot equal zero (0).

The subscripts xₙ and yₘ are integers which represent the number of different possible types of Aᵢ-Bⱼ and Aᵢ-Cₖ constitutional units in a polymer chain, respectively, and p is an integer which represents the average total number of constitutional units in an average polymer chain. Thus, each xₙ is an integer from about 0 to about 100, and yₘ is an integer from about 0 to 150, subject to the constraint that at least one xₙ or at least one yₘ is non-zero. The number of different xₙ groups is a function of the number of different Aᵢ groups and different Bⱼ groups as there is an xₙ for each Aᵢ-Bⱼ group. For example if there are two Aᵢ groups and three Bⱼ groups, there will be six possible Aᵢ-Bⱼ groups (A₁-B₁, A₁-B₂, A₁-B₃, A₂-B₁, A₂-B₂, A₂-B₃), and six xₙ's (x₁, x₂, x₃, x₄, x₅, x₆). The number of different yₘ groups is a function of the number of different Aᵢ groups and different Cₖ groups as there is an yₘ for each Aᵢ-Cₖ group. For example, if there are two Aᵢ groups and three Cₖ groups, there will be six possible Aᵢ-Cₖ groups (A₁-C₁, A₁-C₂, A₁-C₃, A₂-C₁, A₂-C₂, A₂-C₃), and six yₘ's (y₁, y₂, y₃, y₄, y₅, y₆). The average number of constitutional units in a chain, p, is an integer from 2 to about 4500.

Also in the above formula, each of the sᵢ, tⱼ, and vₖ represent the average mole fraction of each of the Aᵢ, Bⱼ, and Cₖ, respectively, which react to form the constitutional units. Each of the sᵢ, tⱼ, and vₖ is a number between 0 and 0.5, inclusive and subject to the constraints that υᵢ sᵢ+Σⱼ tⱼ+Σₖ vₖ=1.0, and Σᵢ sᵢ=Σⱼ tⱼ+Σₖ vₖ=0.5 where each summation of sᵢ is from 1 to the number of different Aᵢ groups (maximum of 10), each summation of tⱼ is from 0 to the number of different Bⱼ groups (maximum of 10), and each summation of vₖ is from 0 to the number of different types of Cₖ groups (maximum of 15). The values are also subject to the limitations that Σᵢ sᵢ>0, and either Σⱼ tⱼ>0 or Σₖ vₖ>0, or there is at least one non-zero sᵢ along with at least one tⱼ or at least one vₖ which is non-zero. Thus, in some embodiments, all vₖ may be 0, or Σₖ vₖ =0, or all tⱼ may be 0 or Σⱼ tⱼ=0, but there are no embodiments where both Σₖ vₖ=0, and Σⱼ tⱼ=0. The mole fraction and the number of constitutional units are obviously related and it is understood that the designation of one will affect the other.

Other than the preceding provisos, sᵢ, tⱼ, and vₖ may be any mole fractions that provide a polymer that exhibits desirable properties for the particular use it is to put as set forth here, e.g., as part of a coating layer for an implantable medical device, subject to the limitations outlined above. However preferred values of vₖ are about 0.1 or less if the Cₖ group is reacted as a free acid (R_{dk}=“H” or hydrogen). Furthermore, it is preferred that the value of vₖ may be low for use with a more hydrophobic drug. Those of ordinary skill in the art will be able to manipulate the mole fractions, prepare the polymers and examine their properties to make the necessary determination based on the disclosures herein without resorting to undue experimentation.

The polymer represented by the above formula may be a random, alternating, random block or alternating block polymer. The term “-/-” means that the Aᵢ-Bⱼ group may be attached to or reacted with another Aᵢ-Bⱼ group, either including the same Aᵢ and Bⱼ or at least one of the Aᵢ and Bⱼ differ, or alternatively, a Aᵢ-Cₖ group. Thus the generic formula encompasses the following exemplary embodiments without limitation. In an exemplary but non-limiting embodiment, if the number of Aᵢ groups is 2 and the number of Bⱼ groups is 2, and the number of Cₖ groups is 1, the following types of polymers are encompassed by the generic formula:

1) . . . -A₁-B₁-A₁-B₂-A₁-C₁-A₁-B₁-A₁-B₂-A₁-C₁-A₁-B₁-A₁-B₂-A₁-C₁-A₁-B₁-A₁-B₂-A₁-C₁— . . . ;

2) . . . -A₁-B₁-A₁-B₁-A₁-B₁-A₂-B₂-A₂-B₂-A₂-B₂-A₁-Cₖ-A₁-Cₖ-A₁-Cₖ-A₁-B₁-A₁-B₁-A₁-B₁-A₂-B₂-A₂-B₂-A₂-B₂-A₁-Cₖ-A₁-Cₖ-A₁-Cₖ-A₁-B₁-A₁-B₁-A₁-B₁-A₂-B₂-A₂-B₂-A₂-B₂-A₁-Cₖ-A₁-Cₖ-A₁-Cₖ— . . . ;

3) . . . -A₁-B₁-A₁-B₁-A₁-B₁-A₁-B₁-A₂-B₂-A₂-B₂-A₂-B₂-A₁-C₁-A₁-C₁-A₂-B₂-A₂-B₂-A₂-B₂-A₂-B₂-A₁-B₁-A₁-B₁-A₁-C₁-A₁-C₁-A₁-B₁-A₁-B₁-A₁-B₁-A₁-B₁-A₁-C₁-A₁-C₁-A₁-C₁-A₁-C₁-A₁-C₁— . . . ;

4) . . . -A₁-B₂-A₂-B₂-A₂-B₂-A₂-B₁-A₂-B₂-A₁-C₁-A₁-B₁-A₂-C₁-A₂-B₂-A₁-C₁-A₂-B₁-A₁-B₁-A₁-C₁-A₂-C₁-A₂-C₁— . . . ;

5) . . . -A₁-B₁-A₁-B₂-A₂-B₁-A₂-B₂-A₁-C₁-A₁-C₂-A₂-B₁-A₂-B₂-A₁-B₁-A₂-C₁-A₁-B₂-A₂-B₂-A₁-B₂-A₂-C₁-A₁-Cₖ-A₂-B₂— . . . .

Thus, there are six potential constitutional units, A₁-B₁, A₁-B₂, A₂-B₁, A₂-B₂, A₁-C₁, and A₂-C₂. As the exemplary embodiments above illustrate, the polymer may be a completely random polymer, a regular alternating polymer, a random alternating polymer, a regular block polymer, or a random block polymer. As illustrated in polymer (2) above, only three groups are included A₁-B₁, A₂-B₂, and A₁-C₁. Such a polymer may be manufactured by reacting the separate blocks and then combining the blocks. Other polymers encompassed by the generic formula contain all six possible constitutional units, such as polymers (4) and (5).

Thus, the generic formula encompasses a polymer with only one type of constitutional unit. If there is only one Aᵢ and only one Bⱼ and no Cₖ groups, there is only the A₁-B₁ unit. If there is only one Aᵢ and no Bⱼ groups and only one Cₖ group, then there is only the A₁-C₁ unit. If there is only one Aᵢ, only one Bⱼ and only one Cₖ group, or only one Aᵢ, only two Bⱼ groups, and not any Cₖ groups, or two Aᵢ groups, and either only one Bⱼ group and not any Cₖ groups, or only one Cₖ group, and not any Bⱼ groups, there will be two potential constitutional units -A₁-B₁ and A₁-C₁ units, A₁-B₁ and A₁-B₂ units, A₁-B₁ and A₂-B₁ units, or A₁-C₁ and A₂-C₁ units, respectively. In general, the total number of potential constitutional units will be equal to the sum of the number of different types of Aᵢ groups times the number of different types of Bⱼ groups, plus the number of different types of Aᵢ groups times the number of different types of Cₖ groups. As outlined above, not all potential constitutional units may be included in each embodiment.

In the above formula, M_{w} represents the weight average molecular weight of the polymer of this invention. Again, while any molecular weight that results in a polymer that has the requisite properties to be used in a coating layer, at present the weight average molecular weight of a polymer of this invention is from about 10,000 Da (Daltons) to about 250,000 Da.

As used herein, “alkyl” refers to a straight or branched chain fully saturated (no double or triple bonds) hydrocarbon (carbon and hydrogen only) group. Examples of alkyl groups include, but are not limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, and cyclohexyl. As used herein, “alkyl” includes “alkylene” groups, which refer to straight or branched fully saturated hydrocarbon groups having two rather than one open valences for bonding to other groups. Examples of alkylene groups include, but are not limited to methylene, —CH₂—, ethylene, —CH₂CH₂—, propylene, —CH₂CH₂CH₂—, n-butylene, —CH₂CH₂CH₂CH₂—, sec-butylene, —CH₂CH₂CH(CH₃)— and the like.

As used herein, “Cm to Cn,” wherein m and n are integers refers to the number of possible carbon atoms in the indicated group. That is, the group can contain from “m” to “n”, inclusive, carbon atoms. An alkyl group of this invention may comprise from 1 to 20 carbon atoms that is m may be 1 and n may be 20. The alkyl group may be linear, branched, or cyclic. Of course, a particular alkyl group may be more limited, for instance without limitation, to 3 to 8 carbon atoms, in which case it would be designate as a (C3-C8)alkyl group. The numbers are inclusive and incorporate all straight or branched chain structures having the indicated number of carbon atoms. For example without limitation, a “C1 to C4 alkyl” group refers to all alkyl groups having from 1 to 4 carbons, that is, CH₃—, CH₃CH₂—, CH₃CH₂CH₂—, CH₃CH(CH₃)—, CH₃CH₂CH₂CH₂—, CH₃CH₂CH(CH₃)— and (CH₃)₃CH—.

As use herein, a “cycloalkyl” group refers to an alkyl group in which the end carbon atoms of the alkyl chain are covalently bonded to one another. The numbers “m” to “n” then refer to the number of carbon atoms in the ring so formed. Thus for instance, a (C3-C8)cycloalkyl group refers to a three, four, five, six, seven or eight member ring, that is, cyclopropane, cyclobutane, cyclopentane, cyclohexane, cycloheptane and cyclooctane.

As used herein,

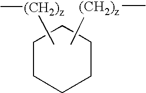

represents a cyclohexane group, optionally with a —CH₂— or a —CH₂—CH₂— group attached at any two locations on the ring, which is the optional groups may be attached at the 1 & 2, 1& 3, or 1 & 4 positions. Alternatively, if z=0, the ring may attach to the other atoms in the molecule at the 1 & 2, 1 & 3, or 1 & 4 positions. Thus the following structures are encompassed:

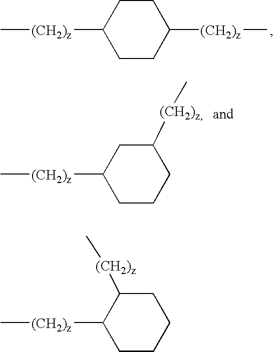

where z is 0, 1, or 2. The conformation of the cyclohexyl groups may be any of the potential conformations which are chair, half-chair, twist boat, or boat. The substituent groups, or the bonds with other molecules, may be either cis or trans.

As used herein, “alkenyl” refers to a hydrocarbon group that contains one or more double bonds.

As used herein, “alkynl” refers to a hydrocarbon group that contains one or more triple bonds.

Standard shorthand designations well-known to those skilled in the art are used throughout this application. Thus the intended structure will easily be recognizable to those skilled in the art based on the required valency of any particular atom with the understanding that all necessary hydrogen atoms are provided. For example, —COR, because carbon is tetravalent, must refer to the structure

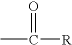

as that is the only way the carbon can be tetravalent without the addition of unshown hydrogen or other atoms.

In some embodiments, the polymer is a PEA random copolymer having the formula:

[(A₁-B₁)ₓ₁|(A₁-B₂)ₓ₂]ₚ(M_{w}, s₁, t₁, t₂)

wherein A₁ has the chemical structure:

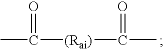

each of B₁ and B₂ has the chemical structure

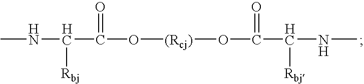

and t₁ is between 0.125 and 0.375, t₂=0.5−t₁, s₁=0.5, and p is an integer from 2 to about 4500. Rₐ₁ is selected from the group consisting of —(CH₂)₆—, —(CH₂)₇—, —(CH₂)₈—, —(CH₂)₉—, and —(CH₂)₁₀—. Each of R_{b1}, R_{b1′}, R_{b2} and R_{b2′} are the same, and are selected from the group consisting of —CH₂—CH(CH₃)₂ and —(CH₃). R_{c1} is selected from the group consisting of —(CH₂)₄—, —(CH₂)₅—, —(CH₂)₆—, —(CH₂)₇—, and —(CH₂)₈—, and R_{c2} is selected from the group consisting of

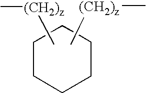

where z is 0, 1, or 2.

In some embodiments, the polymer is one in which Rₐ₁ is —(CH₂)₈—, R_{b1}, R_{b1′}, R_{b2} and R_{b2′} are —(CH₂)—(CH(CH₃)₂), R_{c1} is —(CH₂)₆—; and R_{c2} is

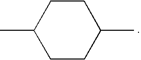

The polymers utilized in the various embodiments of this invention, whether PEA and/or PA polymers or other polymers, may be regular alternating polymers, random alternating polymers, regular block polymers, random block polymers or purely random polymers unless expressly noted otherwise. A representative polymer of x, y, and z constitutional units will be used to illustrate the various types of polymers. To illustrate, a regular alternating polymer has the general structure: . . . x-y-z-x-y-z-x-y-z- . . . . To illustrate, a random alternating polymer has the general structure: . . . x-y-x-z-x-y-z-y-z-x-y- . . . , it being understood that the exact juxtaposition of the various constitution units may vary. To illustrate further, a regular block polymer has the general structure: . . . x-x-x-y-y-y-z-z-z-x-x-x . . . , while an illustrative example of a random block polymer has the general structure: . . . x-x-x-z-z-x-x-y-y-y-y-z-z-z-x-x-z-z-z- . . . . Similarly to the situation above regarding regular and alternating polymers, the juxtaposition of blocks, the number of constitutional units in each block and the number of blocks in block polymers of this invention are not in any manner limited by the preceding illustrative generic structures.

The various embodiments of the present invention include those polymers with a molecular weight in the range of 10,000 to 250,000 Daltons, preferably 70,000 to 150,000 Daltons, and more preferably, 90,000 to 120,000 Daltons.

### Other Polymers

Additional polymers may be utilized with the coating layers of the present invention, or included in an additional coating layer, such as without limitation, a primer layer. Additional polymers may be one or more types. Preferred embodiments utilize biodegradable polymers.

Representative biocompatible polymers include, but are not limited to, poly(ester-amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide/poly(lactic acid) (PEO/PLA)), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as chitosan, alginate, fibrin, fibrinogen, cellulose, starch, dextran, dextrin, fragments and derivatives of hyaluronic acid, polysaccharide, chitosan, alginate, or combinations thereof. Encompassed are also copolymer that include any one of the aforementioned polymers.

As used herein, the terms poly(D,L-lactide), poly(L-lactide), poly(D,L-lactide-co-glycolide), and poly(L-lactide-co-glycolide) can be used interchangeably with the terms poly(D,L-lactic acid), poly(L-lactic acid), poly(D,L-lactic acid-co-glycolic acid), or poly(L-lactic acid-co-glycolic acid), respectively.

### Methods for Enhancing In Vivo Bioabsorption

As outlined above (and in the Examples), it has surprisingly been found that for a coating layer comprising a poly(ester-amide) polymer and a drug, the ratio of the drug to the polymer impacts not only the drug release, but also the in vivo bioabsorption. Furthermore, the in vivo bioabsorption times do not correspond with the in vitro degradation times. It is believed that a similar result will be obtained for the poly(amide) polymers described herein.

Thus various embodiments of the present invention provide methods to fabricate a coated implantable medical device with a coating layer that is bioabsorbed in a given time frame. In some embodiments, the coating layer may be a solid solution while in other embodiments, the coating layer may include a polymer phase of a PEA polymer, a PA polymer, and combinations thereof, and a dispersed drug phase. Additionally, various embodiments of the present invention include methods to enhance or modulate the bioabsorption rate of a coating layer on a substrate, such as an implantable medical device. Modulation includes causing faster and greater water ingress into the coating layer, increasing fraction of interfacial area of the polymer with the dispersed drug phase, and/or increasing the surface area of the coating layer or the interfacial area of the polymer phase with the dispersed drug phase.

### Causing Faster and Greater Water Ingress

In some embodiments of the present invention, modulation of the in vivo bioabsorption of a coating layer may be accomplished by causing the water ingress into the coating layer. The coating layer materials may require exposure to water or fluid for bioabsorption to occur. Thus, causing faster and greater water ingress into the coating layer includes increasing the ratio of drug to polymer. An increase in the rate of water ingress results as drug dissolves on a much shorter time frame than the polymer is absorbed, thus leaving behind pores which allow for water ingress. In other embodiments, the drug and polymer form a solid solution, and increasing the drug to polymer ratio increases the rate of water ingress into the coating layer.

In some embodiments a drug to polymer ratio selected in the range of 1:1 to 1:8, preferably 1:1 to 1:7, and more preferably 1:1 to 1:5, may result in a coating layer that is bioabsorbed in about 12 months or fewer, or a coating layer including a polymer, wherein the polymer is bioabsorbed in about 12 months or fewer.

More generally, as increasing the drug to polymer ratio increases rate of water ingress, the addition of a soluble component, other than the drug, may increase the water ingress into the coating layer. The water diffusion or ingress into the coating layer is enhanced with the addition of a soluble component. Thus, addition of a soluble component may be used to enhance the in vivo bioabsorption rate. In other words, causing faster and greater water ingress into the coating layer includes adding a non-therapeutic soluble component to the coating layer. The resulting coating layer may be a solid solution or comprise a continuous phase and one or more dispersed phases.

Variations in the weight percent of soluble component may impact the in vivo bioabsorption rate, with an increase in weight percent of soluble component leading to an enhancement or increase in the bioabsorption rate. In some embodiments, the drug itself may act as a soluble component. Therefore, some embodiments include the addition of a non-therapeutic soluble component to increase water ingress into the coating layer.

In some embodiments, the non-therapeutic soluble component added may be one with a high osmotic effect. In other words, the addition of such a component increases the fluid or water ingress as the result of the osmotic pressure created by the dissolution of the soluble component in the fluid. Thus, causing faster and greater water ingress into the coating layer also includes adding a soluble component with a high osmotic effect and/or substituting a soluble component (in part or entirely) with another soluble component with a higher osmotic effect.

In some embodiments, the mass ratio of the drug to polymer utilized in the coating layer may be selected to be between about 1:1 to about 1:8 if the drug is a soluble component, or between about 1:1 to about 1:16 if the drug is not categorized as a soluble component. The mass ratio of the soluble components (including drug, if it is a soluble component) to polymer utilized in the coating layer may be selected to be between about 1:1 to about 1:8, preferably 1:1 to 1:7, and more preferably 1:1 to 1:5.

Increasing the Fraction of Interfacial Area of the Polymer with the Dispersed Drug Phase

In some embodiments of the present invention, modulation of the in vivo bioabsorption of a coating layer may be accomplished by increasing the fraction of interfacial area of the polymer with the dispersed drug phase. Thus, as the drug to polymer ratio is increased (or the ratio of soluble components to polymer is increased), the interfacial area between the polymer and the dispersed drug phase increases for the same mass of polymer. For a given mass of polymer there will be a higher fraction of the polymer that is exposed to fluid with a higher drug to polymer ratio, or a higher ratio of soluble components to polymer. As the drug dissolves, pores are left which fill with fluid thus increasing the interfacial area exposed to fluid.

It is believed that the bioabsorption rate constant is higher at the interface between the polymer and the fluid compared to the bulk polymer bioabsorption rate constant, or the overall or effective bioabsorption rate constant. The bioabsorption rate, or chemical degradation constant, may be higher at the interface due to the fact that polymer or materials at an interface generally are in a higher energy state compared to the polymer or material in the bulk. In addition, it is believed that the higher surface area for bioabsorption increases the mass transfer coefficient for the products of absorption, thus providing a larger driving force for further bioabsorption. Thus, the mass transport coefficient may also be influenced by the increased interfacial area as the material at the interface may be in a higher energy state, and this may impact the effective mass transport coefficient. Thus, for a given mass of polymer, the polymer with a higher fraction exposed to fluid has a higher in vivo bioabsorption rate.

Therefore, in some embodiments, increasing the fraction of interfacial area of the polymer with the dispersed drug phase comprises using a drug to polymer ratio in the range of about 1:1 to about 1:8, preferably about 1:1 to about 1:7, and more preferably about 1:1 to about 1:5. In some embodiments, increasing fraction of interfacial area of the polymer with the dispersed drug phase comprises using a soluble component to polymer ratio in the range of about 1:1 to about 1:8, preferably about 1:1 to about 1:7, and more preferably about 1:1 to about 1:5.

Increasing the Surface Area of the Coating Layer or the Interfacial Area of the Polymer Phase with the Dispersed Drug Phase

In some embodiments of the present invention, modulation of the in vivo bioabsorption of a coating layer may be accomplished by increasing the surface area of the coating layer or the interfacial area of the polymer phase with the dispersed drug phase. As noted above, as the fraction of polymer exposed to fluid increases, the in vivo bioabsorption increases. Thus, increasing the surface area of the coating layer or the interfacial area of the polymer phase with the dispersed drug phase increases the rate of in vivo bioabsorption.

Therefore, another method to enhance or impact the in vivo bioabsorption rate is the alteration of the size of the drug and/or soluble component domains. A larger number of smaller domains that are interconnected, or many of which are interconnected, may lead to a higher interfacial surface area once the drug and/or other soluble component has been released. As outlined above, the higher interfacial area is expected to increase the in vivo absorption rate due to increased surface area as well as by virtue of material at the surface having a higher energy state. Similarly, if the combination of the size of the domains of soluble components and the weight percent (or volume percent) of the soluble components in the coating layer is at, or above, the percolation threshold, the in vivo bioabsorption is increased compared to a coating layer in which the soluble components are below the percolation threshold. It is believed that for coating layers in which the soluble components are near the percolation threshold, the in vivo absorption may be greater than those coating layer for which the soluble components are clearly below the percolation threshold.

In some embodiments, the combination of the size of the domains of the soluble components, including the drug if it is categorized as a soluble component, and the weight fraction of the soluble components (or volume fraction) in the coating layer may be altered to insure that the soluble components are present above the percolation limit. Thus, water or fluid can more easily diffuse into the coating layer as the soluble components either diffuse out, or are released from the coating layer. By adjusting the soluble components domain size and/or weight percent in the coating layer such that the soluble components exceed the percolation threshold, it is expected that the in vivo bioabsorption may be enhanced.

In some embodiments, causing faster or greater water ingress includes increasing or selecting the drug and/or soluble components weight fraction and domain size such that the drug and/or soluble components are present at or above the percolation threshold.

In some embodiments, increasing the surface area of the coating layer or the interfacial area of the polymer phase with the dispersed drug phase includes applying the coating layer such that the domain size of the domains of the dispersed drug phase and/or domains of the soluble components are between about 100 nm to 1-2 μm. In other embodiments, increasing the surface area of the coating layer or the interfacial area of the polymer phase with the dispersed drug phase includes applying the coating layer such that the combination of the domain size and volume fraction of the domains of the dispersed drug phase and/or domains of the soluble components are above the percolation threshold. In some embodiments, increasing the surface area of the coating layer or the interfacial area of the polymer phase with the dispersed drug phase includes applying the coating layer such that the domain size of the domains of the dispersed drug phase and domains of the non-therapeutic soluble components, and the volume fractions of the drug and the non-therapeutic soluble components when combined are above the percolation threshold. That a network of interconnected pores is obtained by domains of drug contacting domains of the non-therapeutic soluble substances as well as like domains contacting other like domains.

### Solid Solution

In some embodiments, the coating layer including a PA and/or a PEA polymer and a drug may be a solid solution. In such embodiments, increasing the drug to polymer ratio and/or adding a soluble component also increase water ingress. It is expected that the rate constant for degradation and the mass transfer coefficient will also increase as the drug to polymer ratio increases or with the addition of a soluble component.

### In Vivo Degradation Times

In some embodiments, a method of fabricating a medical device coated with a bioabsorbable coating layer is provided. As outlined above, the range of drug to polymer ratio may be selected in the range of about 1:1 to about 1:8, preferably about 1:1 to about 1:7, and more preferably about 1:1 to about 1:5, or even about 1:3 to about 1:5 in some cases. Alternatively, a ratio of soluble component to drug may be selected in the range of about 1:1 to about 1:8, preferably about 1:1 to about 1:7, and more preferably in the range of about 1:1 to about 1:5. In some embodiments, the soluble component may include a drug, and in some embodiments, the soluble component may not include a drug. Selection of the ratio of soluble component to polymer and/or drug to polymer may be in any of the ranges outlined above.

In any of the embodiments of the present invention, the coating layer thickness may be selected to be between about 2 μm and about 10 μm, or more narrowly between about 4 μm and about 8 μm.

In some embodiments, the coating or coating layer thus fabricated may have an in vivo absorption time of 12 months or fewer, 9 months or fewer, 6 months or fewer, or 3 months or fewer. In some embodiments, about 50% (from 35% to 65%) bioabsorption of the coating or the coating layer has occurred at 3 months post implantation, and in other embodiments, at 6 months post-implantation.

In some embodiments, the coating layer thus fabricated may include a polymer that has an in vivo absorption time of 12 months or fewer, 9 months or fewer, 6 months or fewer, or 3 months or fewer. In some embodiments, about 50% of bioabsorption of the polymer included in coating layer has occurred at 3 months post implantation, and in other embodiments, at 6 months post-implantation.

### Modulation of Coating Layer Absorption by Comparison

A method of fabricating a medical device coated with a bioabsorbable coating layer is provided in some embodiments. A PEA polymer, PA polymer, or combination thereof, and a soluble component, which may be a drug, are selected. Then a coating layer including the PEA polymer, PA polymer, or combination thereof, and the soluble component is applied to an implantable medical device. The ratio of drug to polymer, soluble component to polymer, coating layer thickness, and domain sizes of dispersed drug and/or soluble components are outlined above.

Several different coating layers of different thicknesses and/or compositions may be applied to different substrates of a particular type, such as an implantable medical device, according to the methods outlined above. The in vivo bioabsorption of the different coating layers on the different substrates may be measured.

In some embodiments, the in vivo absorption of a bioabsorbable coating layer on a substrate may be modulated. The modulation involves first applying to a substrate a coating layer comprising a PEA polymer, PA polymer, or combination thereof, and a drug at a first soluble component to polymer mass ratio, and at a selected coating layer thickness. The in vivo absorption rate of the coating layer with the first soluble component to polymer mass ratio is determined. A second coating layer is applied to a second substrate where the second coating layer includes a PEA polymer, PA polymer, or combination thereof, and a drug at a second selected soluble component to polymer mass ratio, and at the same selected coating layer thickness used for the first coating layer layer. The determination of the in vivo absorption rate of the coating layer with the second soluble component to polymer mass ratio is made. The determination of the in vivo absorption rates may occur sequentially, with either the first or the second coating layer measured first in time, at the same time, or in the same experimental protocol. In some embodiments, the second coating layer may be applied to a substrate before the in vivo absorption of the first coating layer has been determined.

After the in vivo bioabsorption rates of the two coating layers have been made, a graph can be made. The graph is made by plotting the in vivo absorption as an absolute mass loss of either the coating layer, or the polymer included in the coating layer, over a specific time period on the abscissa; versus the fraction of polymer in the coating layer on the ordinate. A straight line may be drawn between the points on the graph. Then, one may select a desired in vivo absorption time, and determine the soluble component to polymer mass ratio and the coating layer thickness to obtain the desired in vivo absorption time.

The use of the graph would require iteration. In other words, one would select a soluble component to polymer ratio, determine the coating layer thickness or amount of polymer in the coating layer by mass, and then estimate the in vivo absorption time using the mass loss of polymer (or coating layer) over a specified time period obtained from the graph for the given soluble component to polymer mass ratio. If the estimate is too high, that is the estimated bioabsorption time is longer than desired, a higher soluble component to polymer mass ratio may be chosen, smaller domain sizes for the drug and/or soluble components may be used, and/or the coating layer thickness may be reduced. The process would then be repeated until the estimated bioabsorption time was sufficiently close, such as, without limitation, within 10%-20%, of the desired bioabsorption time.

In some embodiments utilizing the above graphical procedure, the second coating layer may utilize the same polymer and the same drug as were utilized in the first coating layer. In other embodiments both the polymer and the drug may differ from the first to the second coating layers. In still other embodiments, only one of the polymer or the drug may be the same for the first and second coating layers. In any of the above embodiments, non-therapeutic soluble components may be included, or may be excluded. In the event that a non-therapeutic soluble component is included in both the first and second coating layers, the same or a different non-therapeutic soluble component may be utilized for each of the two coating layers. In some embodiments, only one of the two coating layers may include a non-therapeutic soluble component. In some embodiments, the coating layers used for making the graph may not include a drug, but a PEA polymer, PA polymer, or combination thereof, and a soluble component are included the coating layer. Thus, the same procedures may be used as those outlined above for coating layers including a PEA polymer, PA polymer, or combination thereof, and a drug.

Thus, some embodiments of the present invention include a method for fabricating, and if necessary, modulating, the in vivo absorption of a bioabsorbable coating layer on a substrate. A coating layer including a PEA polymer, PA polymer, or combination thereof, and a soluble component which may be a drug, are applied to a substrate. The soluble component to polymer mass ratio is selected to be between about 1:2 and about 1:10, and the coating layer thickness applied is between about 2 μm and 10 μm. The in vivo bioabsorption may be determined, and if not acceptable, may be modulated. An acceptable bioabsorption may be within about 30%, preferably 20%, and more preferably 10%, of an objective or desired bioabsorption time. If the in vivo bioabsorption rate is too fast, it may be decreased by decreasing the soluble component to polymer ratio, increasing the coating layer thickness, increasing the domain size of the drug and/or soluble component, or a combination thereof. If the in vivo bioabsorption rate is too slow, it may be increased by increasing the soluble component to polymer ratio, decreasing the coating layer thickness, decreasing the domain size of the drug and/or soluble component, or a combination thereof.

In any of the embodiments of the present invention, the bioabsorption rate may also be increased by substituting a non-therapeutic soluble component with a different non-therapeutic soluble component which has a higher osmotic potential than the initial non-therapeutic soluble component. In some embodiments, only part of the non-therapeutic soluble component may be replaced with a non-therapeutic soluble component with a higher osmotic potential.

### Coating Constructs

In some embodiments, the coating on the implantable medical device such as a stent will contain only one layer that is the coating layer including the poly (ester-amide) and/or poly(amide) polymer. A coating refers to one or more coating layers.

In some embodiments, there may be additional coating layers above or below the coating layer including the poly (ester-amide) or poly(amide) polymer. There may be any number of coating layers (including 0 or none) below the coating layer including a PEA polymer, a PA polymer, or a combination thereof, and any number of coatings (including 0 or none) above the coating layer including a PEA polymer, a PA polymer, or a combination thereof. There may be more than one coating layer including a PEA polymer, a PA polymer, or a combination thereof, with any number of layers between them (including 0 or none).

Preferred embodiments include a coating with only one layer which is the layer including a PEA polymer, a PA polymer, or a combination thereof.

In any of the above embodiments, any of the layers, including the optional primer layer, the coating layer including the PEA and/or PA polymer, any optional layers above the coating layer including the PEA and/or PA polymer, and any optional layers intervening between the primer layer and the coating layer including the PEA and/or PA polymer layer may optionally include one or more drugs.

In any of the above embodiments, any of the layers, the coating layer(s) including the PEA and/or PA polymer, any optional layers above, below, or between these coating layers may be applied over all, or substantially all, of the outer surface of the substrate, or only over a portion of the surface, such as a selected portion of the surface. For a stent, the outer surface includes both the luminal and abluminal surfaces. A non-limiting example of a selected portion for a stent may be the luminal side only.

### Method of Use

In accordance with embodiments of the invention, the coating and/or coating layers according to the present invention can be included in an implantable device or prosthesis, e.g., a stent. For a device including one or more drugs, the drugs will be retained on the device such as a stent during delivery and expansion of the device, and released at a desired rate and for a predetermined duration of time at the site of implantation.

For implantation of a stent, an angiogram is first performed to determine the appropriate positioning for stent therapy. An angiogram is typically accomplished by injecting a radiopaque contrasting agent through a catheter inserted into an artery or vein as an x-ray is taken. A guidewire is then advanced through the lesion or proposed site of treatment. Over the guidewire is passed a delivery catheter that allows a stent in its collapsed configuration to be inserted into the passageway. The delivery catheter is inserted either percutaneously or by surgery into the femoral artery, brachial artery, femoral vein, or brachial vein, and advanced into the appropriate blood vessel by steering the catheter through the vascular system under fluoroscopic guidance. A stent having the above-described coating may then be expanded at the desired area of treatment. A post-insertion angiogram may also be utilized to confirm appropriate positioning.

### Examples of Implantable Devices

The coatings and/or coating layers of these embodiments can be applied to any medical devices where the bioabsorption of the coating layer in about 12 months or fewer is necessary or desirable. The underlying structure of the device can be of virtually any design. Particularly suitable medical devices are implantable medical devices. A preferred device for use with the various embodiments of the present invention is a stent.

As used herein, an “implantable medical device” refers to any type of appliance that is totally or partly introduced, surgically or medically, into a patient's body (human or veterinary patient) or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, artificial heart valves, cerebrospinal fluid shunts, and intrauterine devices. An implantable medical device specifically designed and intended solely for the localized delivery of a therapeutic agent is within the scope of this invention.

Other medical devices that may be used with the various embodiments of the present invention include catheters, endocardial leads (e.g., FINELINE™ and ENDOTAK,™ available from Abbott Cardiovascular Systems Inc., Santa Clara, Calif.), and devices facilitating anastomosis such as anastomotic connectors.

A type of implantable medical device is a “stent.” A stent refers generally to any device used to hold tissue in place in a patient's body. Particularly useful stents, however, are those used for the maintenance of the patency of a vessel in a patient's body when the vessel is narrowed or closed due to diseases or disorders including, without limitation, tumors (in, for example, bile ducts, the esophagus, the trachea/bronchi, etc.), benign pancreatic disease, coronary artery disease, carotid artery disease and peripheral arterial disease such as atherosclerosis, restenosis and vulnerable plaque. Vulnerable plaque (VP) refers to a fatty build-up in an artery thought to be caused by inflammation. The VP is covered by a thin fibrous cap that can rupture leading to blood clot formation. A stent can be used to strengthen the wall of the vessel in the vicinity of the VP and act as a shield against such rupture. A stent can be used in, without limitation, neuro, carotid, coronary, pulmonary, aorta, renal, biliary, iliac, femoral and popliteal as well as other peripheral vasculatures. A stent can be used in the treatment or prevention of disorders such as, without limitation, thrombosis, restenosis, hemorrhage, vascular dissection or perforation, vascular aneurysm, chronic total occlusion, claudication, anastomotic proliferation, bile duct obstruction and ureter obstruction.

The device may be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable and/or biostable polymers could also be used with the embodiments of the present invention. The device can be, for example, a bioabsorbable stent.

### EXAMPLES

The examples presented in this section are provided by way of illustration of the current invention only and are not intended nor are they to be construed as limiting the scope of this invention in any manner whatsoever. Each of the examples the follows relates to the coating of 3 mm×12 mm VISION™ (Abbott Cardiovascular Systems Inc.) stent, which has a coatable surface area of 0.5556 cm².

### Example 1

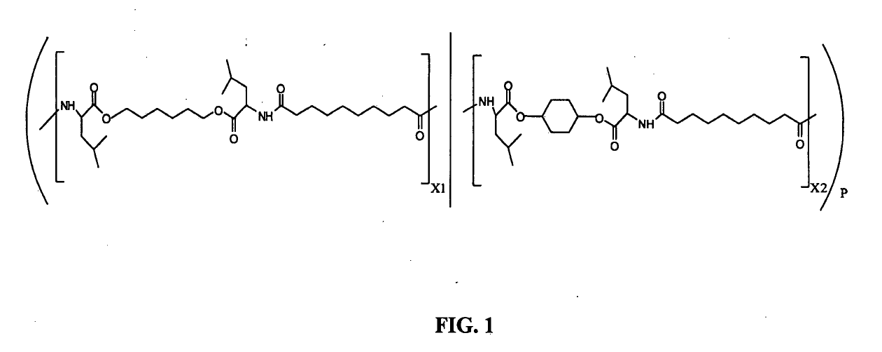

All stents were cleaned by being sonicated in isopropyl alcohol, followed by an argon plasma treatment. Stents were coated with a coating layer of the poly(ester-amide) polymer of FIG. 1 (“PEA 40”) and everolimus (supplied by Novartis) by spraying from a solution of ethanol (absolute). The polymer in FIG. 1 is a random copolymer of the two constitutional units, X1 and X2, where the p indicates a polymer or multiple functional units. The “/” indicates that the two constitutional units are randomly arranged. The PEA polymer was manufactured by standard methods. The PEA polymer, PEA 40, was purified and reprecipitated several times, and there were no detectable levels, or essentially no detectable levels, of residual reactants or catalyst in the polymer. The PEA polymer utilized had a glass transition of 52-56° C., a molecular weight of 100-200 kD, and a polydispersity of 1.5-2.0.

Two mass ratios of drug to polymer were utilized, 1:3 and 1:5, sprayed from solution of about 1-2 weight percent solids. The spraying operation was carried out with a custom made spray coater equipped with a spray nozzle, a drying nozzle, and a means to rotate and translate the stent under the nozzles with the processing parameters outlined in Table 1. Multiple passes under the coating and drying nozzle were required to obtain the target weight of polymer and drug on the stent. After the drug layer coating, the stents were baked in a forced air convection oven at 50° C. for 1 hour. After baking the coating, the stents were crimped onto 3.0×12 mm XIENCE V catheters, placed in protective tubular coils, and then sealed in Argon filled foil pouches. Sterilization was performed by e-beam irradiation. A simulated use test followed sterilization. The simulated use test involves expanding the stent in poly(vinyl alcohol) tube which simulates a vessel, and then followed by exposure to a flow of 37° C. distilled water flowing through the stent for 1 hour. The water flow rate is 50 ml/min.

TABLE 1
Spray Processing Parameters Coating
Coating Parameters
Spray Head

| | |
|---|---|
| Spray nozzle to stent distance (mm) | 12 ± 1 |
| Solution flow rate | 2-4 ml/hr |
| Atomization pressure (psi) | 12.5 ± 0.5 |

Air Dry Heat Nozzle

| | |
|---|---|
| Drying nozzle temp (° C.) | 50-80 |
| Drying nozzle pressure (psi) | 15 ± 2 |
| Spray nozzle to stent distance (mm) | 12 ± 1 |

Flow Rate and Coating Weight

| | |
|---|---|
| Target Flow Rate in μg/pass | 7-14 |
| Coating Weight Pre-Bake (μg) | Variable |
| Coating Weight Post-Bake (μg) | Variable |
| Target Weight (μg) | Variable |

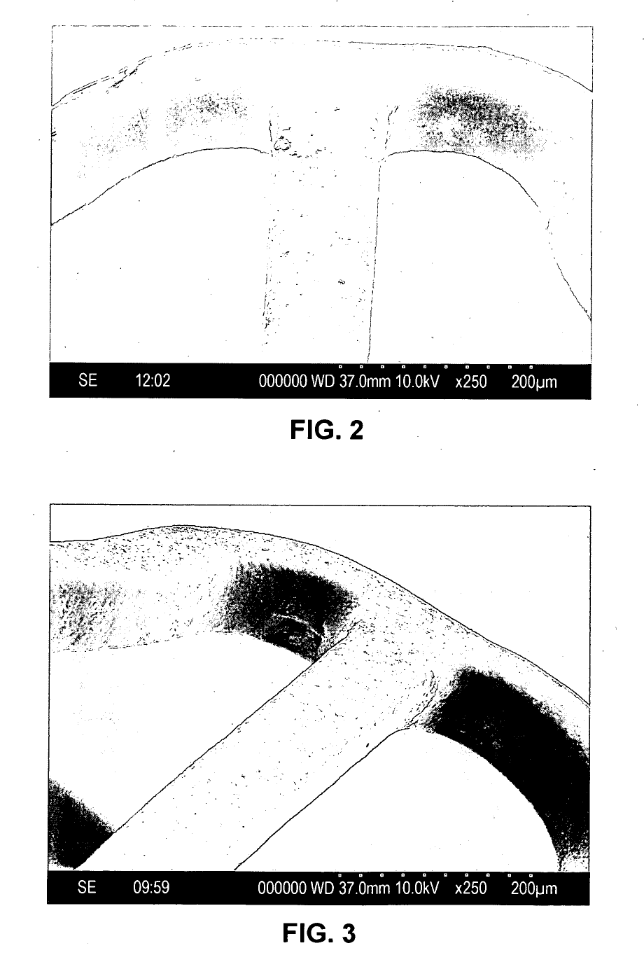

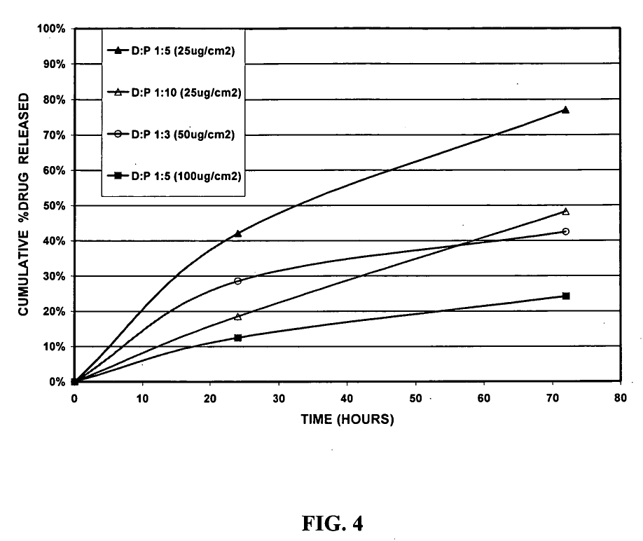

FIGS. 2 and 3 are depictions of the coated stents after the simulated use test.

### Example 2

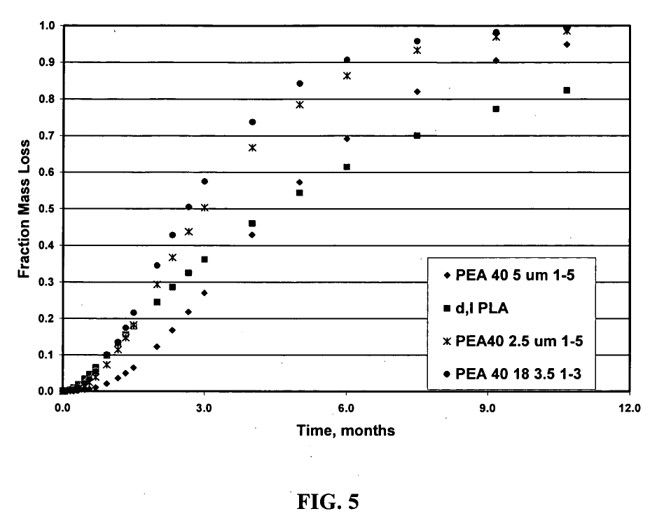

Stents were coated as outlined in Example 1. The same polymer and drug were utilized, but different drug to polymer mass ratios, and different thickness coating layers were investigated. These stents were not sterilized. Cumulative release of the drug everolimus was determined using a United States Pharmacopeia type 7 tester with dissolution media of porcine serum with sodium azide 0.1% (w/v) added. Everolimus released into solution was determined by HPLC analysis on the amount of drug remaining on the stent. The cumulative release expressed is one minus the fractional amount of drug remaining divided by the theoretical quantity, or dose, of drug per stent that is expressed as a percent. The cumulative release of drug released for n=3 stents is illustrated in FIG. 4. As illustrated in FIG. 4, the variations in drug to polymer mass ratio (“D:P” in FIG. 4) and coating layer thickness (expressed in FIG. 4 as mass everolimus/cm²) results in different cumulative release profiles.

### Example 3

Stents were coated as outlined in Example 1. The same polymer and drug were utilized. An in vivo bioabsorption experiment was carried out with three different coating configurations illustrated in Table 2 below. The in vivo bioabsorption experiment utilized pigs. Two stents were implanted per pig in different arteries. The control was the Xience™ drug eluting stent. After a designated time period, the animals were euthanized and a section of the artery including the stent was removed. The entire artery section including the stent was placed in a solvent, such as chloroform, which extracted the polymer. Polymer molecular weight was determined with Gel Permeation Chromatography using a polystyrene standard. Mass loss was determined by a calibration curve for the GPC.

TABLE 2
Coating Layers Evaluated in In vivo Experiment

| | A | B | C |
|---|---|---|---|
| Everolimus dose μg/cm² | 100 | 100 | 50 |
| Drug to polymer mass ratio | 1:5 | 1:3 | 1:5 |
| Coating Layer thickness, μm | 5.0 | 3.5 | 2.5 |
| Polymer mass, μg | 280 | 168 | 140 |

The in vivo absorption data is summarized in Tables 3 and 4 below. The mass loss % quantifies the mass of the polymer lost due to in vivo degradation. The loss of drug is assumed as the drug is released on a time frame much shorter than polymer degradation. M_{w} refers to the weight-average molecular weight of the polymer and the acronym “PDI” refers to the polydispersity index of the polymer which is the ratio of the weight-average molecular weight to the number average molecular weight.

TABLE 3
In vivo Degradation of Various Coating Layers

| | A | | | B | | | C | | |
|---|---|---|---|---|---|---|---|---|---|
| Time- | Mass | M_{w} | | Mass | M_{w} | | Mass | M_{w} | |
| point | Loss % | (kD) | PDI | Loss % | (kD) | PDI | Loss % | (kD) | PDI |
| 0 | 0 | 100.5 | 1.72 | 0 | 130.8 | 1.65 | 0 | 100.5 | 1.72 |
| 3- | 23.6 ± 14.3 | 86 | 1.57 | 55.8 ± 8.8 | 95.7 | 1.48 | 51.0 ± 12.4 | 76.6 | 1.56 |
| months | | | | | | | | | |
| 6- | 72.1 ± 18.6 | 55.6 | 1.8 | TBD | TBD | TBD | TBD | TBD | TBD |
| months | | | | | | | | | |

TABLE 4
In vivo Degradation of Various Coating Layers expressed as Absolute
Mass Lost

| | Absolute Mass Loss of Polymer, μg | | | | | |
|---|---|---|---|---|---|---|
| Time-Point | A | | B | | C | |
| 0 months | 0 | μg | 0 | μg | 0 | μg |
| 3 months | 66.1 ± 40.0 | μg | 93.7 ± 14.8 | μg | 71.4 ± 17.4 | μg |

Although the amount of mass lost is approximately the same for different thickness coating layers at the same drug to polymer ratio, the rate is expected to be modestly higher for a thinner coating layer due to the fact that the transport of by-products from the coating layer is quicker. However, the use of the drug to polymer ratio alone is sufficient initially to estimate the in-vivo absorption.

### Example 4

Stents were coated following the same procedures used for Example 1. The same polymer and drug, everolimus, were utilized. The drug to polymer mass ratio was 1:5, and the everolimus dose was 100 μg/cm². The coating layer thickness was 5 μm, and the coating layer included 280 μg of polymer. The stents were analyzed for in vitro degradation by being placed in water or phosphate buffer system (PBS), placed in an oven at 37° C., and removed and analyzed at the time-points. The analysis followed the same procedure as described in Example 3, which is solvent extraction and GPC analysis. The results of the in vitro degradation are shown in Table 5 below.

As shown in Table 5, there was very little mass loss of polymer in the in vitro test.

TABLE 5
In vitro Degradation

| Time Point | Mw | Mass Recovery |
|---|---|---|
| (weeks) | (Daltons) | (%) |
| 0 | 100500 | 100 |
| 3-month | 96323 | 98 |
| 6-month | 79572 | 97 |

### Example 5

The mass loss determined from the experiments performed and described in Example 3 were modeled along with the in vivo bioabsorption of a coating layer composed of D,L polylactide. The model was an approximate mechanistic absorption model that is a mechanistic model which approximates the absorption processes for the coating layers. The two major parameters were the chemical rate constant and the mass transfer rate constant. The results of the modeling are shown in FIG. 5. As depicted in FIG. 5, the coating layers including a poly(ester-amide) polymer are expected to bioabsorb within 12 months.

### Example 6

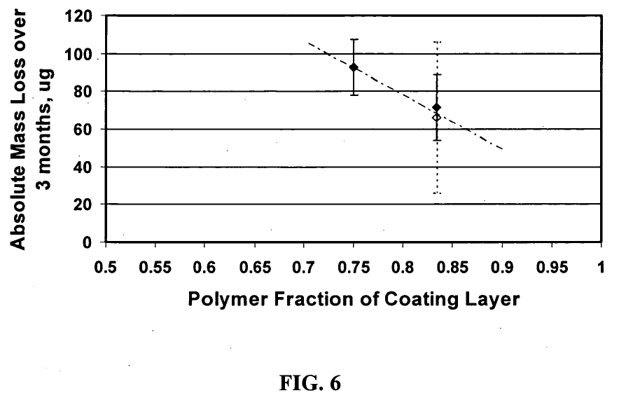

The mass loss determined from the experiments performed and described in Example 3 is plotted in FIG. 6. On the abscissa is the absolute mass loss over three months in μg, and the polymer mass fraction in the coating layer is plotted along the ordinate. The open diamond point corresponds to the dashed error bars. The data included in FIG. 6 includes data for coating layers of various thickness. The line in FIG. 6 is least-squares linear fit to the in vivo data. Thus, from the graph in FIG. 6, a polymer volume fraction of 0.8 (or D:P of 1:4) is estimated to show 80 μg mass loss due to in vivo absorption over 3 months.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A method of modulating the in vivo absorption rate of a coating on an implantable medical device comprising a poly(ester-amide) (PEA), a poly(amide) (PA) polymer, or a combination thereof, and a drug, the coating layer comprising a polymer phase and a dispersed drug phase, the method comprising:
a) causing faster and greater water ingress into the coating layer,
b) increasing fraction of interfacial area of the polymer with the dispersed drug phase, and/or
c) increasing the surface area of the coating layer or the interfacial area of the polymer phase with the dispersed drug phase;
wherein the coating layer completely degrades or substantially degrades within 12 months after implantation of the implantable medical device.

2. The method of claim 1, wherein the method modulates the in vivo absorption rate of a coating layer on an implantable medical device, and wherein the implantable medical device is a stent.

3. The method of claim 1, wherein the coating layer completely or substantially degrades within 6 months after implantation.

4. The method of claim 1, wherein the coating layer has degraded by about 50% at 3 months after implantation.

5. The method of claim 1, wherein causing faster and greater water ingress into the coating layer comprises adding a non-therapeutic soluble component to the coating layer.

6. The method of claim 1, wherein increasing the fraction of interfacial area of the polymer with the dispersed drug phase comprises using a drug to polymer ratio in the range of about 1:1 to about 1:7.

7. The method of claim 1, wherein increasing fraction of interfacial area of the polymer with the dispersed drug phase comprises using a soluble component to polymer ratio in the range of about 1:1 to about 1:7.

8. The method of claim 1, wherein increasing the surface area of the coating layer or the interfacial area of the polymer phase with the dispersed drug phase comprises applying the coating layer such that the domain size of the domains of the dispersed drug phase are between about 100 nm and about 2 μm.

9. The method of claim 1, wherein the method modulates a coating layer on an implantable medical device, and wherein the coating layer comprises a poly(ester-amide) polymer, a poly(amide) polymer, or a combination thereof, and a drug, and wherein the poly(ester-amide) or poly(amide) polymer is a polymer of the formula: 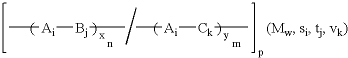
wherein:
i is an integer from 1 to 10, inclusive;
j is an integer from 0 to 10, inclusive;
k is an integer from 0 to 15, inclusive;
xₙ is an integer from 0 to 100, inclusive;
yₘ is an integer from 0 to 150, inclusive;
p is an integer from 2 to about 4500;
M_{w} is from about 10,000 to about 1,000,000 Da;
sᵢ is a number from 0 to 0.5, inclusive;
tⱼ is a number from 0 to 0.5, inclusive;
vₖ is a number from 0 to 0.5, inclusive;
with the proviso that
Σᵢ sᵢ+Σⱼ tⱼ+Σₖ vₖ=1.0;
Σᵢ sᵢ=Σⱼ tⱼ+Σₖ vₖ=0.5;
Σᵢ sᵢ>0;
Σⱼ tⱼ>0 or Σₖ vₖ>0;
each Aᵢ has the chemical structure: 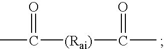
each Bⱼ has the chemical structure 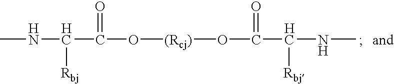
each Cₖ has the chemical structure: 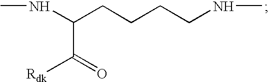
wherein:
each R_{bj}, and R_{bj′} are independently selected from the group consisting of hydrogen and (C1-C4)alkyl, wherein:
the alkyl group is optionally substituted with a moiety selected from the group consisting of —OH, —SH, —SeH, —C(O)OH, —NHC(NH)NH₂, 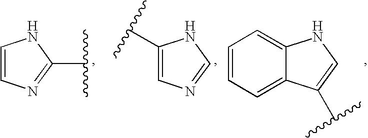
phenyl and 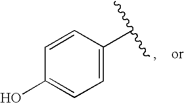
one or more of R_{bj} and R_{bj′} may form a bridge between the carbon to which it is attached and the adjacent nitrogen, the bridge comprising —CH₂CH₂CH₂—;
each Rₐᵢ and each R_{cj} are independently selected from the group consisting of (C1-C12)alkyl, (C2-C12)alkenyl, (C3-C8)cycloalkyl, —(CH₂CH₂O))_{q}CH₂CH₂— wherein q is an integer from 1 to 10, inclusive, and 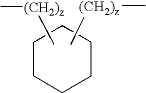
where z is 0, 1, or 2;
R_{dk} is selected from the group consisting of —H, —OH, —O(C1-C20)alkyl, —O(C1-C20)alkenyl and —O(CH₂CH₂O)_{w}CH₂CH₂ORₑₖ, wherein:
w is an integer from 1 to 600, inclusive;
Rₑₖ is selected from the group consisting of hydrogen, —C(O)CH═CH₂ and —C(O)C(CH₃)═CH₂; and,
each Rₐᵢ corresponds to the i^{th} Aᵢ group, each R_{bj}, R_{bj′}, and R_{cj} corresponds to the j^{th} Bⱼ group, and each R_{dk} and optionally Rₑₖ correspond to the k^{th} Cₖ group.

10. The method of claim 9, wherein for the polymer i=1, j=2, k=0, and
each of Rₐ₁ is selected from the group consisting of —(CH₂)₆—, —(CH₂)₇—, —(CH₂)₈—, —(CH₂)₉—, and —(CH₂)₁₀—;
each of R_{b1}, R_{b1′}, R_{b2} and R_{b2′} are the same, and are selected from the group consisting of —(CH₂)—(CH(CH₃)₂) and —(CH₃);
R_{c1} is selected from the group consisting of —(CH₂)₄—, —(CH₂)₅—, —(CH₂)₆—, —(CH₂)₇—, and —(CH₂)₈—; and
R_{c2} is selected from the group consisting of 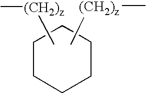
where z is 0, 1, or 2.

11. A method of fabricating an implantable medical device coated with a bioabsorbable coating layer, the method comprising:
applying to an implantable medical device a coating layer comprising a poly(ester-amide) and/or a poly(amide) polymer, and a soluble component;
wherein the soluble component to polymer ratio is between 1:1 to 1:7; and
wherein the coating layer thickness is between about 2 μm and 10 μm; and
wherein the polymer in the coating layer, or the coating layer, is substantially absorbed or completely absorbed in vivo in 12 months or fewer after implantation.

12. The method of claim 11, wherein the coating layer comprises a drug.

13. The method of claim 11, wherein the coating layer comprises a non-therapeutic soluble component.

14. The method of claim 11, wherein the polymer is a random poly(ester amide) copolymer having the formula:
[(A₁-B₁)ₓ₁|(A₁-B₂)ₓ₂]ₚ(M_{w}, s₁, t₁, t₂)
wherein:
A₁ has the chemical structure: 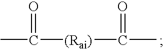
each of B₁ and B₂ has the chemical structure 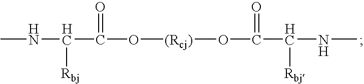
t₁ is between 0.125 and 0.375;
t₂=0.5−t₁;
s1=0.5; and
p is an integer from 2 to about 4500;
wherein:
Rₐ₁ is selected from the group consisting of —(CH₂)₆—, —(CH₂)₇—, —(CH₂)₈—, —(CH₂)₉—, and —(CH₂)₁₀—;
each of R_{b1}, R_{b1′}, R_{b2} and R_{b2′} are the same, and are selected from the group consisting of —(CH₂)—(CH(CH₃)₂) and —(CH₃);
R_{c1} is selected from the group consisting of —(CH₂)₄—, —(CH₂)₅—, —(CH₂)₆—, —(CH₂)₇—, and —(CH₂)₈—; and
R_{c2} is selected from the group consisting of 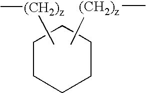
where z is 0, 1, or 2.

15. A coating layer on a substrate, the coating layer comprising:
a drug and a poly(ester-amide) polymer and/or a poly(amide) polymer wherein the ratio of drug to polymer is from about 1:3 to about 1:5;
wherein the coating layer thickness on the substrate is between 2 μm to about 10 μm; and
wherein the polymer mass in the coating layer after 3 months is about 50% or less of the initial polymer mass in the coating layer wherein the reduction is due to in vivo absorption.

16. The coating layer of claim 15, wherein the polymer is a random poly(ester-amide) copolymer having the formula:
[(A₁-B₁)ₓ₁|(A₁-B₂)ₓ₂]ₚ(M_{w}, s₁, t₁, t₂)
wherein:
A₁ has the chemical structure: 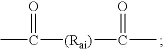
each of B₁ and B₂ has the chemical structure 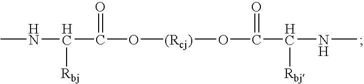
t₁ is between 0.125 and 0.375;
t₂=0.5−t₁;
s1=0.5; and
p is an integer from 2 to about 4500;
wherein:
Rₐ₁ is selected from the group consisting of —(CH₂)₆—, —(CH₂)₇—, —(CH₂)₈—, —(CH₂)₉— and —(CH₂)₁₀—;
each of R_{b1} R_{b1′}, R_{b2} and R_{b2′} are the same, and are selected from the group consisting of —(CH₂)—(CH(CH₃)₂) and —(CH₃);
R_{c1} is selected from the group consisting of —(CH₂)₄—, —(CH₂)₅—, —(CH₂)₆—, —(CH₂)₇—, and —(CH₂)₈—; and
R_{c2} is selected from the group consisting of 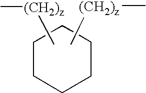
where z is 0, 1, or 2.

17. An implantable medical device comprising a coating layer of claim 16.

18. The device of claim 17 wherein the implantable medical device is a stent.

19. The coating layer of claim 16, wherein for the poly(ester-amide) polymer
Rₐ₁ is —(CH₂)₈—;
R_{b1}, R_{b1′}, R_{b2} and R_{b2′} are —(CH₂)—(CH(CH₃)₂);
R_{c1} is —(CH₂)₆—; and
R_{c2} is

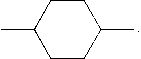

20. A method of fabricating an implantable medical device coated with a bioabsorbable coating layer, the method comprising:
providing an implantable medical device;
applying to the implantable medical device a coating layer comprising a poly(ester-amide) and/or a poly(amide) polymer, and a soluble component;
wherein the soluble component to polymer ratio is between 1:1 to 1:7; and
wherein the coating layer thickness is between about 2 μm and 10 μm;
determining the in vivo bioabsorption time of the coating layer;
wherein if the in vivo bioabsorption time is too long:
increasing the ratio of soluble component to polymer,
decreasing the coating layer thickness, and/or
decreasing the domain size of the soluble component;
or if the in vivo bioabsorption time is too short:
decreasing the ratio of soluble component to polymer,
increasing the coating layer thickness, and/or
increasing the domain size of the soluble component.

21. The method of claim 19, wherein the soluble component comprises a drug.

22. The method of claim 19, wherein the soluble component comprises a nontherapeutic soluble component selected from the group consisting of poly(vinyl pyrrolidone), poly(ethylene glycol) (PEG), poly(propylene glycol), and combinations thereof.

23. The method of claim 20, wherein the polymer is a random poly(ester-amide) copolymer having the formula:
[(A₁-B₁)ₓ₁|(A₁-B₂)ₓ₂]ₚ(M_{w}, s₁, t₁, t₂)
wherein:
A₁ has the chemical structure: 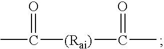
each of B₁ and B₂ has the chemical structure 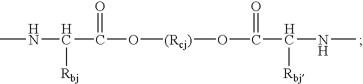
t₁ is between 0.125 and 0.375;
t₂=0.5−t₁;
s1=0.5; and
p is an integer from 2 to about 4500;
wherein:
Rₐ₁ is —(CH₂)₈—;
R_{b1}, R_{b1′}, R_{b2} and R_{b2′} are —(CH₂)—(CH(CH₃)₂);
R_{c1} is —(CH₂)₆—; and
R_{c2} is [chemical structure]
